(19) United States
(12) Patent Application Publication
(10) Pub. No.: US 2024/0245022 A1
(43) Pub. Date: Jul. 25, 2024

(54) Hydrangea plant 'HMOWH05-0'

(71) Applicant: GreenFuse Botanicals llc, Santa Paula, CA (US)

(72) Inventor: James Devereux, Fort Collins, CO (US)

(73) Assignee: GreenFuse, Santa Paula, CA (US)

(21) Appl. No.: 18/099,903

(22) Filed: Jan. 20, 2023

(51) Int. Cl. A01H 6/48; A01H 5/02

(52) U.S. Cl. CPC A01H 6/48; A01H 5/02

(57) ABSTRACT

The invention relates to the field of Hydrangea, specifically, the variety designated ‘HMOWH05-0’. The variety ‘HMOWH05-0’ is characterized by its lack of vernalization requirement for induction and development of flowering, day-length neutral flowering, cold tolerance to USDA Zone 5 and lace-cap inflorescence having white sterile flowers and pink fertile flowers. The present invention relates to plant parts, including cells and any propagative material of the new variety ‘HMOWH05-0’, and use of any of the plant parts for reproducing the new variety ‘HMOWH05-0’. The present invention relates to methods using any plant parts or progeny of ‘HMOWH05-0’ for the purpose of deriving additional new Hydrangea varieties. The present invention relates to seed, plants and plant parts produced by crossing ‘HMOWH05-0’ with any other Hydrangea variety or other plant. The present invention also relates to methods to produce new varieties of Hydrangea using the variety ‘HMOWH05-0’ and applying plant breeding techniques.

### FIELD OF THE INVENTION

The present invention relates to a new variety of Hydrangea referred to as ‘HMOWH05-0’ as well as to new, distinct and stable characteristics found in Hydrangea. The present invention relates to plants which have all of the morphological and physiological characteristics described herein, as well as plant parts which can be used to reproduce plants having the characteristics specific to Hydrangea referred to as ‘HMOWH05-0’. The present invention also relates to methods for producing these plants of Hydrangea exhibiting the characteristics described herein. Furthermore, the present invention relates to a method of producing progeny Hydrangea plants by crossing the Hydrangea referred to as ‘HMOWH05-0’, as either the female or seed or male or pollen parent, with another Hydrangea plant, or ‘HMOWH05-0’, and selecting progeny. The present invention also relates to methods to produce new varieties of Hydrangea using the variety ‘HMOWH05-0’ in a breeding program.

### BACKGROUND OF THE INVENTION

The present invention relates to a new, distinct and stable variety of Hydrangea, hereinafter referred to by the variety denomination ‘HMOWH05-0’. The new Hydrangea ‘HMOWH05-0’ originated from a controlled breeding program in Santa Paula, CA.

Hydrangea belong to the family Hydrangeacea and is a Genus consisting of about 25 species which are native to the Western Hemisphere and Eastern Asia. Hydrangea are a widely distributed ornamental shrub or subshrub. Extensive breeding programs exist for Hydrangea worldwide. Methods for propagation are well known and include terminal vegetative cuttings.

Hydrangea is one of the most important Genus of commercial flowering ornamental plants. Hundreds of varieties, both patented and unpatented are available to the public. Demand for and supply of Hydrangea plants has increased significantly in the last 20 years in the commercial horticulture market. Ornamental breeders seek to improve upon known characteristics as well as develop new and useful features.

When grown for ornamental horticulture, vernalization is required to induce flowering in Hydrangea plants. This process adds significantly to the cost and time of commercial plant production. It is the objective of the breeder to create efficiencies with improved varieties, both in terms of financial crop inputs and total crop time.

The current market of Hydrangea varieties all require vernalization in order to be brought into bloom. Significant efficiency can be realized by eliminating this step for the commercial grower.

Most known commercial varieties of Hydrangea bloom under long day conditions. As consumers prefer to buy flowering plants, this limits the sales period to Spring and Summer, unless artificial light conditions are provided to plants.

The new variety eliminates the need for vernalization. This represents significant potential to reduced propagative inputs for growers as well a shorter period of time to bring a plant into bloom. The day-length neutral characteristic increases the sales season, without the need to manipulate photoperiod.

Objectives of breeding programs are defined by the problems and weaknesses of the current cultivars. Crossing and selection have been made to improve flowering induction characteristics in Hydrangea. The variety described herein is an ornamental Hydrangea plant, having the above-mentioned improvements combined with attractive ornamental characteristics. Plants retain the desirable ornamental traits of Hydrangea, including large, colorful inflorescences, remontant flowering, together with novel and improved floral induction.

To those trained in the art, the improvements described herein are both valuable and quantitative assets.

### SUMMARY OF THE INVENTION

The following embodiments and aspects thereof are described in conjunction with systems, tools and methods which are meant to be exemplary, not limiting in scope.

The present invention provides Hydrangea plant selections exhibiting no need for vernalization to begin flowering. Additionally, plants of ‘HMOWH05-0’ begin flowering under any day-length, being day-length neutral. Plants of Hydrangea ‘HMOWH05-0’ are hardy to USDA Zone 5. These qualities, combined with the description included herein, distinguish the new cultivar from known Hydrangea varieties.

These and other improvement and objectives have been achieved in accordance with the present invention which provide ‘HMOWH05-0’ as a new Hydrangea cultivar that is the product of a planned breeding program conducted by the inventor in Santa Paula, CA.

The new variety ‘HMOWH05-0’ can be produced by asexual reproduction to produce a population of progeny plants, each of which has the combination of characteristics as herein disclosed for the new variety ‘HMOWH05-0’.

### Definitions

Terminology below may be useful for description and understanding of the new invention.
Day-Length: A common term referring to the hours of daylight required to induce and develop flowering. Syn. photoperiodism.
Day-Length Neutral: A flowering condition in which neither short nor long days are required to induce and develop flowering.
Dormant: Temporarily inactive.
First Node: The first growth zone above the roots on the stem.
Hortensia: A common name in usage for Hydrangea
Morphology: Form and structure.
Node: Growth zone.
Plant form: Overall structure and shape of the plant.
Progeny: Off-spring or descendants.
Vernalization: The cooling of plant material in order to induce flowering.

Additional Terminology, Related to Polyploidy
Euploids—polyploids with exact multiples of the complete set of chromosomes specific to a species. Haploid or Monoploid (one set; 1×), Diploid (two sets; 2×), Triploid (three sets; 3×), Tetraploid (four sets; 4×), Pentaploid (five sets; 5×), Hexaploid (six sets; 6×), Octaploid (eight sets; 8×), Decaploid (ten sets; 10×), Dodecaploid (12 sets; 12×).
Autopolyploids—polyploids with multiple chromosome sets derived from a single species—usually derived from chromosome doubling.
Allopolyploids—polyploids whose chromosomes are derived from different species. are a combination of genomes of different species (Acquaah 2007) due to hybridisation of two or more genomes followed by chromosome doubling or by the fusion of unreduced gametes between species (Acquaah 2007; Chen 2010; Jones et al. 2008; Ramsey and Schemske 1998). Can accure naturally or artificially
Aneuploids—polyploids that contain either an addition or deletion of one or more specific chromosome(s) to the total number of chromosomes that usually make up the ploidy of a species (Acquaah 2007; Ramsey and Schemske 1998). Aneuploids are formed due to the formation of univalents and multivalents during meiosis of euploids

### DEPOSIT INFORMATION

Biological material which is the variety ‘HMOWH05-0’ is to be deposited with Bigelow Laboratory for Ocean Sciences, East Boothbay, ME, a Budapest Treaty recognized depository which affords permanence of the deposit. Upon receipt the accorded International Depository Authority Accession Number will be provided as necessary.

### BRIEF DESCRIPTION OF THE DRAWINGS

The patent or application file contains multiple drawings executed in color. Copies of this patent or patent application publication with color drawing will be provided by the Office upon request and payment of the necessary fees.

The accompanying photographs illustrate the overall appearance and morphology of the new Hydrangea ‘HMOWH05-0’ showing the colors as true as is reasonably possible with colored reproductions of this type. Colors in the photograph may differ slightly from the color values cited in the detailed botanical description which accurately describes the color of ‘HMOWH05-0’.

FIG. 1 illustrates a side view of plants of Hydrangea ‘HMOWH05-0’ at approximately 26 to 28 weeks of age. This photo was taken in Somis CA.

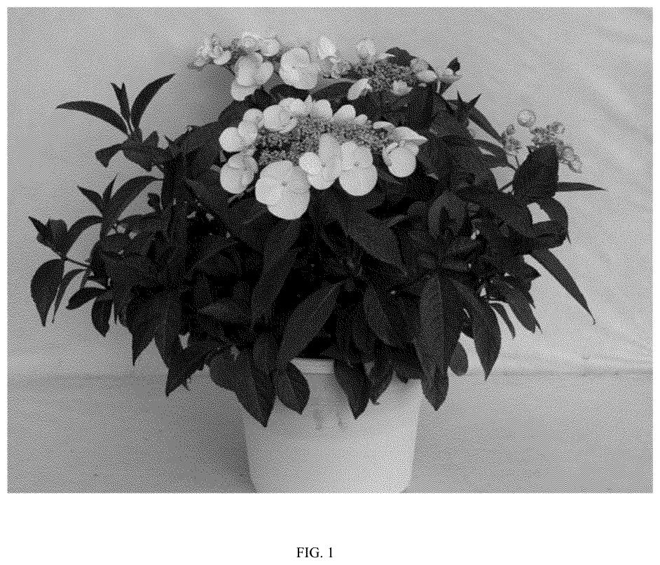

FIG. 2 illustrates a close-up view of flowers of Hydrangea ‘HMOWH05-0’

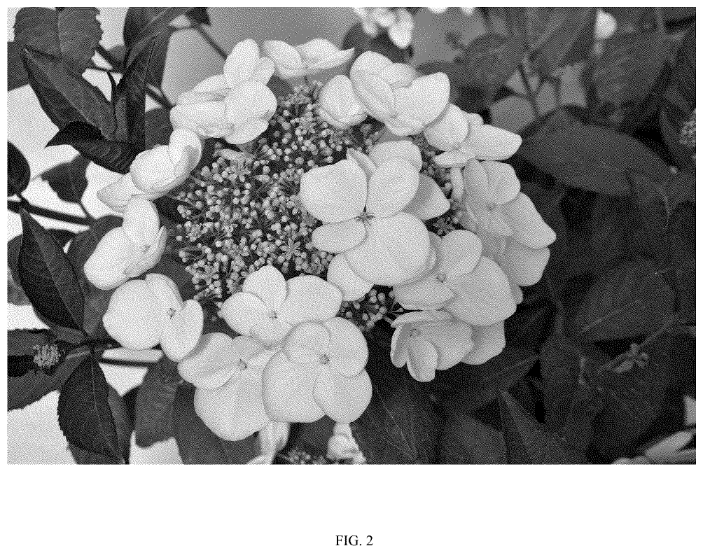

### OBJECTS OF THE INVENTION

The following embodiments and aspects thereof are described in conjunction with system, tools and methods which are meant to be exemplary, not limiting in scope

The present invention relates to Hydrangea plants, and parts thereof, having all the physiological and morphological characteristics of Hydrangea ‘HMOWH05-0’.

Another embodiment relates to a plant produced by vegetative means which are Hydrangea ‘HMOWH05-0’. The present invention also relates to plant parts, such as pollen, seeds or inflorescence produced by Hydrangea ‘HMOWH05-0’.

Another embodiment relates to a method of producing seed which are Hydrangea ‘HMOWH05-0’.

Another embodiment also relates to a method of producing Hydrangea progeny comprising the steps of (a) self-pollinating Hydrangea ‘HMOWH05-0’ (b) harvesting seeds produced from said self-pollination; and (c) producing plants from said harvested seeds.

The present invention also relates to producing progeny plants from the cross of Hydrangea ‘HMOWH05-0’, as the female or male parent, with another Hydrangea plant, and selecting progeny plants from this cross.

The present invention also relates to producing progeny plants of Hydrangea ‘HMOWH05-0’, by any known means of vegetative propagation.

The present invention also relates to producing progeny plants from Hydrangea ‘HMOWH05-0’, from natural or induced mutation.

Another embodiment relates to tissue culture produced from protoplast of cells from Hydrangea ‘HMOWH05-0’ plants disclosed in the subject application, wherein said cells or protoplasts are produced from a plant part selected from the group consisting of pollen, ovules, embryos, protoplasts, meristematic cells, callus, leaves, anthers, cotyledons, hypcotyl, pistils, roots, root tips, flowers, seeds, petiole and stems.

Another embodiment relates to a plant or a part thereof, produced by growing Hydrangea ‘HMOWH05-0’, wherein the plant part comprises at least one cell of Hydrangea ‘HMOWH05-0’.

Another embodiment relates to tissue or cell culture of regenerable cells produced from the plants of Hydrangea ‘HMOWH05-0’ as well as a Hydrangea plant regenerated from the tissue or cell culture of Hydrangea ‘HMOWH05-0’.

Another embodiment relates to a method of vegetatively propagating the plant of Hydrangea ‘HMOWH05-0’, comprising the steps of: collecting tissue or cells capable of being propagated from a plant of Hydrangea ‘HMOWH05-0’; cultivating said tissue or cells to obtain proliferated shoots; and rooted said shoots to obtain rooted plantlets; or cultivating said tissue or cells to obtain shoots or to obtain plantlets and a plant produced by growing the plantlets or shoots of said plant.

A further embodiment relates to a method for developing an Hydrangea plant in an Hydrangea breeding program, comprising applying plant breeding techniques comprising crossing, recurrent selection, mutation breeding, wherein said mutation breeding selects for a mutation that is spontaneous or artificially induced, backcrossing, pedigree breeding, marker enhanced selection, haploid/double haploid production, ploidy manipulation, or transformation to the Hydrangea plant of Hydrangea ‘HMOWH05-0’, or its parts, wherein application of said techniques results in development of an Hydrangea plant.

A further embodiment relates to a method of introducing a mutation into the genome of Hydrangea ‘HMOWH05-0’, and wherein the resulting plant comprises at least one genome mutation and producing plants there from.

In addition to the exemplary aspects and embodiments described above, further aspects and embodiments will become apparent by study of the following descriptions.

### BRIEF DESCRIPTION OF THE INVENTION

This invention is directed to Hydrangea plants having all the morphological and physiological characteristics of the variety ‘HMOWH05-0’ produced from either vegetative cuttings or tissue culture.

The new Hydrangea ‘HMOWH05-0’ can be produced by asexually reproducing progeny. Asexual reproduction of the new cultivar by vegetative means was first performed by vegetative cuttings during May of 2018, in Santa Paula, CA. The first ‘HMOWH05-0’ plants propagated through the use of such cuttings are maintained in Santa Paula, CA and have reproduced multiple generations. Subsequent asexual reproduction has demonstrated that the new cultivar reproduces true-to-type and that the combination of characteristics as herein disclosed for the new cultivar are firmly fixed and retained through successive generations of asexual reproduction.

The following traits have been repeatedly observed and are determined to be unique characteristics of ‘HMOWH05-0’ which in combination distinguish these Hydrangea as new and distinct cultivars:
1. No vernalization required for induction and development of flowering.
2. Flowering is day length neutral and remontant.
3. Cold tolerant to USDA Zone 5.
4. Lace-cap inflorescence having white sterile flowers and pink fertile flowers.

Hydrangea ‘HMOWH05-0’ can also be compared to Hydrangea ‘HMOPICO04-0’, U.S. utility patent application Ser. No. 17/401,099. Plants of ‘HMOPICO04-0’ differ from plants of ‘HMOWH05-0’ in the following:
1. Plants of the new variety produce white sepals on the sterile flowers; plants of ‘HMOPICO04-0’ produce white sepals with a distinctive red-pink margin on the sterile flowers.
2. Fertile flowers of the new variety are brighter pink than fertile flowers of this comparator.

Hydrangea ‘HMOWH05-0’ can also be compared to Hydrangea ‘HMOPI02-0’, U.S. utility patent application Ser. No. 17/401,088. Plants of ‘HMOPI02-0’ differ from plants of ‘HMOWH05-0’ in the following:
1. Plants of the new variety produce white sepals on the sterile flowers; plants of ‘HMOPI02-0’ produce pink sepals on the sterile flowers.

‘HMOWH05-0’ has not been tested and observed under all possible environmental conditions. The phenotype of the new cultivar may vary with variations in environment such as temperature, light intensity, frequency of fertilization, composition of fertilizer, flowering treatment, day length and humidity, without any change in the genotype of the plant.

For example, substantial differences in plant height and diameter can be observed, depending upon environmental conditions and planting density. Inflorescence size and color can vary as well.

### DETAILED BOTANICAL DESCRIPTION

The aforementioned drawings, together with the following observations, measurements and values describe the new Hydrangea ‘HMOWH05-0’ as grown in a greenhouse in Santa Paula, CA. Plants of ‘HMOWH05-0’ were grown in a research greenhouse with temperatures ranging from approximately 5° C. to 18° C. during the day and night temperatures ranging from approximately 20° C. to 30° C. in the day and approximately 15° C. to 25° C. in the day. No artificial lighting or photoperiodic treatments were conducted. Plants were measured at approximately 4.5 months old from an unrooted cutting.

Color reference are made to the Royal Horticultural Society Colour Chart (RHS), 2007 edition, except where general colors of ordinary significance are used. Color values were taken under daylight conditions in a greenhouse in Ventura County, CA.

### Plant

Plant type: Flowering deciduous perennial shrub.
Age of Plant Described: Approximately 4.5 months from an unrooted cutting.
Growth Habit: Broadly rounded.
Height: 55 cm.
Plant Spread: 65 cm.
Plant vigor: Moderate.
Branching Habit: Strong branching from a pinch. Branches occurring at mostly obtuse angles.
Length of Primary Lateral Branches: Average range 30 to 45 cm.
Diameter of Lateral Branches: 5 to 6 mm.
Quantity of Lateral Branches: Average range 16 to 24.
Stem:
   Stem shape: Round.
   Stem strength: Strong.
   Color:
      Immature: RHS Green 137A flushed Greyed-Purple 187A.
      Mature: RHS Green 137A.
   Texture: Glabrous.
   Aspect: Straight, occurring at 15° to 75° from center.
Internode length: 5 to 7 cm.

### Foliage

Leaf:
   Arrangement: Opposite.
   Leaf shape: Elliptic.
   Average Length: 7 to 11 cm.
   Average Width: 3 cm.
   Apex: Acuminate.
   Base: Obtuse.
   Margin: Dentate.
   Texture of top surface: Glabrous.
   Texture of bottom surface: Glabrous.
   Color:
      Young foliage upper side: Emerging foliage near Greyed-Purple 187A.
      Young leaves near Green 137A, flushed Greyed-Purple 187A. Margin flushed Greyed-Purple N187A.
      Young foliage under side: Emerging foliage near Greyed-Purple 187B.
      Young leaves near Green 137C, flushed Greyed-Purple 187A. Margin flushed Greyed-Purple N187A.
      Mature foliage upper side: RHS Green 137C
      Mature foliage under side: RHS Green 137D.
   Venation:
      Pattern: Pinnate.
      Color upper side: RHS Yellow-Green 144A.
      Color under side: RHS Yellow-Green 144B.
   Petiole:
      Average Length: Average range 1.5 to 2.5 cm.
      Diameter: 3 mm to 4 mm.
      Petiole color upper side:
         Young foliage RHS Greyed-Purple 187B.
         Mature foliage: RHS Green 137C, edges flushed Greyed-Purple 187B.
      Petiole color lower side:
         Young foliage RHS Greyed-Purple 187B.
         Mature foliage: RHS Green 137C, edges flushed Greyed-Purple 187B.
      Petiole Texture upper side: Glabrous.
      Petiole Texture lower side: Glabrous.

### Flower

Natural flowering season: Day length neutral, flowering begins approximately 13 to 16 weeks after planting during any time of the year without chill hours.
Inflorescence:
   Type: Lacecap. Compound umbel.
   Quantity of flowers per inflorescence:
      Fertile flowers: 300-400.
      Sterile flowers: Average 25.
   Shape: Flattened disc.
   Height: About 10 to 14 cm.
   Diameter: 15 cm to 22 cm.
   Fragrance: None detected.
Sterile Flower:
   Bud shape: Broad ovate.
   Bud length: 15 mm.
   Bud diameter: 10 mm.
   Bud color: Near RHS White N155C flushed Red 55D.
   Aspect: Upright.
   Flower depth: 8 mm.
   Flower Diameter: 4 to 6 cm.
   Persistence: Self-cleaning.
Sterile Flower Petals: Petals do not develop/open, or not present.
Sterile Flower Sepal:
   Arrangement: Cruciform, moderately overlapping.
   Number: 4.
   Shape: Wide deltate to irregular orbicular.
   Tip: Broad acute.
   Base: Obtuse.
   Margin: Entire.
   Length: 2.5 to 3.7 cm.
   Width: 3 to 4 cm.
   Texture, Upper: Smooth.
   Texture, Lower: Smooth.
   Color:
      Upper surface at first opening: Near RHS White N155D, very thin margin coloration near Red 54C.
      Under surface at first opening: Near RHS White N155D, very thin margin coloration near Red 54C.
      Upper surface at maturity: Near RHS White N155D.
      Under surface at maturity: Near RHS White N155D, very thin margin coloration near Red 54C sometimes present.
Sterile Flower Pedicel:
   Length: 2.5 cm to 3.5 cm.
   Diameter: 3 mm.
   Angle: About 75°, pedicel mostly straight.
   Strength: Flexible.
   Texture: Glabrous.
   Color: Immature near RHS Red 54C; Mature near Yellow-Green 145C.
Fertile Flower: Rotate flower composed of petals and noticeably protruding stamens.
   Bud shape: Sphere
   Bud length: 4 mm.
   Bud diameter: 3 mm.
   Bud color: Near RHS Yellow-Green 150C, turning Red 55D just before opening.
   Flower aspect: Upright.
   Flower shape: Rotate.
   Flower diameter: 5 to 7 mm.
   Flower length: 4 mm petals only; including protruding stamens 6 mm
   Persistence: Self-cleaning.
Fertile Flower Petals:
   Arrangement: Rotate.
   Quantity: 5.
   Length of petal: 2 to 3 mm.
   Width of petal: 1 mm to 2 mm.
   Apex: Acute.
   Shape of petal: Narrowly elliptic.
   Petal margin: Entire.
   Petal base: Truncate.
   Petal Texture: Smooth, upper and under side.
   Color:
      Upper surface at first opening: Near Red 55B.
      Under surface at first opening: Near Red 55C.
      Upper surface at maturity: Near Red 55C.
      Under surface at maturity: Near Red 55D.
Fertile Flower Sepal: Minute and inconspicuous, less than 0.5 mm, colored near Red 55C.
Fertile Flower Pedicel:
   Angle: About 45° to 75°.
   Strength: Firm.
   Length: Average 5 mm.
   Width: 1 mm.
   Texture: Glabrous.
   Color: Near RHS Yellow-Green 145C flushed Red 54C.

### Reproductive Organs

Sterile flowers: Androecium irregularly present. Somewhat mis-formed gynoecium irregularly present.
Stamens:
   Number: 4 to 6.
   Filament length: 5 mm.
   Filament color: RHS Greyed-Red 182D.
Anthers:
   Shape: Globular.
   Length: 1 mm.
   Color: RHS Red 54D.
   Pollen color: Not observed.
Pistil:
   Number: 3.
   Length: 2 to 3 mm.
   Style:
      Length: 1 to 3 mm.
      Color: Near Red 55C.
   Ovary: About 0.5 mm in diameter and length. Colored near Red 55D
   Stigma:
      Shape: Bi-lobed.
      Color: Near Red-Purple 65D.
Fertile flowers:
Stamens:
   Number: About 8 to 10.
   Filament length: 5 mm to 9 mm.
   Filament color: RHS Red-65C.
Anthers:
   Shape: Globular.
   Length: 1 mm.
   Color: RHS Green-White 157D.
   Pollen color: RHS Yellow-White 158D.
   Pollen amount: Scant.
Pistil:
   Number: 3.
   Length: 3 mm.
   Style:
      Length: 2 mm.
      Color: Near Red 55C.
   Ovary color: Near Red 55D.
   Stigma:
      Shape: Bi-lobed.
      Color: Near Red 55B.

### OTHER CHARACTERISTICS

Seeds and fruits: Not observed to date.

Disease/pest resistance: Neither resistance nor susceptibility to normal diseases and pests of Hydrangea have been observed.

Temperature tolerance: Low temperature tolerant to USDA Zones 5.

We claim:

1. A plant of Hydrangea variety ‘HMOWH05-0’, representative biological material to be deposited at Bigelow Laboratory for Ocean Sciences, East Boothbay, ME.

2. A plant or a plant part thereof produced by growing the plant of claim 1, wherein the plant or plant part comprises at least one cell of Hydrangea ‘HMOWH05-0’.

3. A Hydrangea plant or part thereof, having the physiological and morphological characteristics of the plant of claim 1.

4. A tissue or cell culture of regenerable cells produced from the plant of claim 1.

5. The tissue or cell culture of claim 4, comprising tissues or cells from a plant part selected from the group consisting of leaves, vegetative cuttings, pollen, embryos, cotyledons, hypocotyl, meristematic cells, roots, root tips, pistils, anthers, flowers and stems.

6. A method of producing Hydrangea progeny comprising the steps of (a) crossing a plant of Hydrangea ‘HMOWH05-0’ as a female or male parent with another Hydrangea plant or other plant and (b) selecting progeny.

7. The method according to claim 6, wherein the second Hydrangea plant is another plant of Hydrangea ‘HMOWH05-0’.

8. A method for developing a Hydrangea plant in a plant breeding program using plant breeding techniques, applied to a pant of Gypsophila variety DGYPSALBBF’ or a part thereof; the method comprising a technique selected from crossing, recurrent selection, backcrossing, pedigree breeding, market enhanced selection, haploid production, double haploid production, transformation, mutation breeding, wherein said mutation breeding selects for a mutation that is spontaneously or naturally induced or artificially induced, thereby developing a Hydrangea plant derived from the variety ‘HMOWH05-0’.
